(19) United States
(12) Patent Application Publication
(10) Pub. No.: US 2005/0249698 A1
(43) Pub. Date: Nov. 10, 2005

(54) Synergistic effect of the osteogenic growth peptide and the granulocyte clony stimulating factor on haematogenesis

(76) Inventors: Zhihui Liu, Shanghai (CN); Dafu Cui, Shanghai (CN); Zhongwei Chen, Shanghai (CN); Deyuan Shi, Shanghai (CN); Yi Lu, Shanghai (CN)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 10/526,028

(22) PCT Filed: Sep. 16, 2002
(86) PCT No.: PCT/CN02/00660

§ 371 Date: Feb. 28, 2005

(30) Foreign Application Priority Data
Aug. 28, 2002 (CN) 02136688.8

(51) Int. Cl. A61K 38/19; A61K 38/08; A61K 38/10

(52) U.S. Cl. 424/85.1; 514/13; 514/14; 514/15; 514/16

(57) ABSTRACT

The present inventions provides a pharmaceutical composition comprising a safe and efficient amount of OGP, a safe and efficient amount of G-CSF and a pharmaceutically acceptable carrier, wherein the molar ratio of OGP to G-CSF is from 0.25:1 to 100:1. According to the present invention, there is a good synergism between OGP and G-CSF. Thus the haematogenesis of G-CSF is greatly promoted. The invention also discloses the preparation method and usage of the pharmaceutical composition, especially in promoting the haematogenesis of G-CSF and the immune response of lymphocyte.

### FIELD OF INVENTION

The present invention relates to medical field, and, in particular, to the synergistic effect of osteogenic growth peptide (OGP) and granulocyte colony-stimulating factor (G-CSF) on haematogenesis, and the pharmaceutical composition comprising OGP and G-CSF.

### PRIOR ART

In 1988, Itai Bab, et al., discovered osteogenic growth peptide (OGP), a polypeptide of 14 amino acids which improved the growth of the osteocytes. OGP is originated from 14-peptide at the C-terminal of histone H4, that is the processed product resulting from the translation initialing from AUG85 at mRNA level after the transcription of the gene of histone H4 [Bab I, et al. (1999) J. Biol. Chem. 274(20): 14474-14481]. During the regeneration of the marrow, several osteogenesis promoting factors are released and enter into the blood circulation, which enhance the systematic ossification. Osteogenic growth peptide was obtained by isolation and purification [Bab I, et al. (1988) Endocrinology 123:345-352; Bab I, et al. (1992) EMBO J. 11: 1867-1873].

The OGP sequences of the OGPs presented in human and mice serum are completely identical and possess same biological activity [Greenberg Z, et al. (1995) J. Clin. Endocrinol Metab 8: 2330-2335]. Under physiological conditions, OGPs are presented in bound form, that is, OGP-OGP binding protein (OGPBP) complex, which accounts for about 80%-97% of total amount of OGP [Greenberg Z, et al. (1995) JCE & M. 80(8): 2330-2335]. OGPBP in serum is α2 macroglobulin, whose function is perhaps to protect serum OGP from degradation, or regulate the level of active OGP in the serum [Gavish H, et al. (1997) Biochemistry 36: 14883-14888]. OGP C-terminal 5-peptide may be the enzymatic digestion product during the dissociation of OGP and its binding protein, which is also presented in nature and has many similar activities as OGP [Bab I, et al. (1999) J Pept Res 54: 408-414].

Synthetic OGP (sOGP) has completely identical sequence like the nature OGP, which can in vitro promote the propagation of osteoblasts, fibroblasts and human marrow stroma cells, the activity of the alkaline phosphatase of osteoblasts, human and rabbit bone marrow stroma cells, and in vivo promote the bone formation and the weight of bone trabecula in rat [Bab I., et al. (1992) EMBO J. 11: 1867-1873; Robinson D. et al. (1995) J. Bone and Mineral Research 10(5): 690-696].

OGP is not only osteogenic but also facilitates haematogenesis. OGP can promote the increase of the numbers of peripheral blood leukocytes and bone myeloid cell. Administration of sOGP to radiation impaired mice before bone marrow transplantation can help the reestablishment of haematogenesis and increase mice survival rate [Gurevitch O, et al. (1996) Blood 88(12): 4719-4724]. The administration of OGP C-terminal 5-peptide to bone marrow impaired mice by cyclophosphamide can accelerate the recovery of mice peripheral blood leukocytes and recruit mice peripheral blood stem cells [Rita F, et al. (2002) Leukemia Research 19-27]. However, the effect of OGP on haematogenesis is quite weak, significantly weaker than granulocyte colony stimulating factor (G-CSF) and granulocyte-macrophage colony stimulating factor (GM-CSF).

G-CSF is clinically used and commercially available, such as filgrastim originated from the E. coli non-glycosylated recombinant protein, lenograstim originated from the glycosylated molecular of CHO (Chinese hamster ovary cell), nartograstim which is N-terminal substituted [Maruyama K, et al. (1998) Bone Marrow Transplant 22(4): 313-320]. The main biological activities of G-CSF are promoting the propagation of granuloprogenitor cells and differentiation into neutrophilic leukocytes, and improving the survival of mature neutrophilic leukocytes and the well establishment of the function thereof, including phagocytolysis, sterilization and the cytotoxic effect mediated by antibodic cells [Ohsaka A, et al. (1998) Br J Haematol 100(1): 66-69]. Recent research showed that, for failed acute myeloblastic leukemia, chronic myeloblastic leukemia, foreign bone marrow transplantation after using united pretreatment protocol including systematic radiation, large dosage of cytosine arabinoside and G-CSF in haematogenic progenitor crisis period can reduce the recurrence rate and improve the disease-free survival rate without serious unwanted response [Takahashi M, et al. (1997) Am J Hematol 56(1): 42-44; Takahashi S, et al. (1998) Am J Hematol 57(4): 303-308]. At present, G-CSF is also used to recruit peripheral blood stem cells for autograft and allograft. The recruitment protocol most commonly used is using G-CSF after cyclophosphamide or using G-CSF alone. However, because the haematogenesis stem cells and progenitor cells (CD34⁺ cells) recruited by these two protocols have potential to develop into different clones, the different recruitment protocols should be used for different purposes [Cesana C, et al. (1998) Bone Marrow Transplant 21(6): 561-568].

At present, many growth factors are found to improve the recruitment of mice peripheral blood stem cells in high dosage chemotherapy group or normal group, which can be used in peripheral blood stem transplantation, or increase the amount of bone marrow karyote and restore the amount of peripheral blood leukocytes. These factors include granulocyte colony stimulating factor (G-CSF), granulocyte-macrophage colony stimulating factor (GM-CSF), interleukin-3 (IL-3), stem cell factors (SCF), Flt-3 ligands, etc. [Bungart B, et al. (1990) Br J Haematol 76(2): 174-179; Lane T, et al. (1995) Blood 85(1): 275-282; Brugger W, et al. (1992) Blood 79(5): 1193-1200; Molineux G, et al. (1991) Blood 78(4): 961-966; Ashihara E, et al. (1998) Eur J Haematol 60(2): 86-92]. Recombinant human granulocyte colony stimulating factor (rhG-CSF) and/or recombinant human granulocyte-macrophage colony stimulating factor (rhGM-CSF) are mainly used clinically to recruit the peripheral blood stem cells or promote the recovery of haematogenesis after radiotherapy/chemotherapy. However, these two methods are very expensive and are hardly affordable by ordinary people. Furthermore, there are some problems in clinical use. High dosage of rhG-CSF may cause unwanted response such as osteodynia. High dosage of rhGM-CSF will be accompanied by fever. Moreover, when using them to recruit peripheral blood stem cells for peripheral blood stem cell transplantation, it can hardly obtain enough peripheral blood stem cells for transplantation in one administration course, which brings pain to the stem cell donor and make the receptor wait till the next stem cell transplantation, thus increasing the risk. IL-8, IL-11, SCF, Flt-3 ligand or macrophage inflammation protein-1α(MIP-1α) can also recruit the peripheral stem cells, but weaker than G-CSF or GM-CSF [Andrews R G, et al. (1992) Blood 80: 920-927; Haas R, et al. (1993) 12: 643-649; Lemoli R M, et al. (1993) 21: 1668-1672; Jacoben S E, et al. (1995) J Exp Med 181: 1357-1363; Laterveer L, et al. (1996) 87: 781-788; Hunter M G, et al. (1995) 86: 4400-4408].

Therefore, it is a need of inexpensive haematogenesis promoting pharmaceutical compositions in art.

### SUMMARY OF INVENTION

One purpose of the invention is to provide an inexpensive pharmaceutical composition for promoting hemopoiesis.

Another purpose of the invention is to provide a method for preparing the pharmaceutical composition.

In the first aspect, the invention provides a pharmaceutical composition comprising a safe and efficient amount of osteogenic growth peptide or OGP, a safe and efficient amount of granulocyte colony-stimulating factor or G-CSF, and a pharmaceutically acceptable carrier, wherein the molar ratio of OGP to G-CSF is from 0.25:1 to 100:1.

In one preferred embodiment, the pharmaceutical composition further comprises a component selected from the group consisting of GM-CSF, EPO, IL-2, or the combination thereof.

In another preferred embodiment, the OGP is selected from the group consisting of human OGP, OGP-related peptide, their pharmaceutically acceptable salts, and the combination thereof.

In another preferred embodiment, the molar ratio of OGP to G-CSF is from 1:1 to 20:1.

In another preferred embodiment, the formulation of the pharmaceutical composition is injection, or lyophilized powder.

In the second aspect, the invention provides a method for preparing a pharmaceutical composition, comprising the following steps:
mixing the OGP, G-CSF and a pharmaceutically acceptable carrier, thereby obtaining the pharmaceutical composition, wherein the molar ratio of OGP to G-CSF is from 0.25:1 to 100:1.

In the third aspect, the invention provides the use of the mixture of OGP and/or OGP-related peptide together with rhG-CSF for preparing a pharmaceutical composition for promoting the hemopoiesis of rhG-CSF.

### BRIEF DESCRIPTION OF THE DRAWINGS

FIG. 1 shows the effect of co-administration of sOGP and rhG-CSF to normal mice after administration of sOGP for 5 days alone on white blood cell (WBC) number in different time.

#: the comparison of co-administration group and the group using rhG-CSF alone, p<0.0005

*: the comparison of co-administration group and the group using rhG-CSF alone, p<0.05

FIG. 2 shows the effect of co-administration of sOGP and rhG-CSF to normal mice after administration of sOGP for 5 days alone on leukocytes (LY) number in different time.

#: the comparison of co-administration group and the group using rhG-CSF alone, p<0.0005

*: the comparison of co-administration group and the group using rhG-CSF alone, p<0.05

FIG. 3 shows the pathologic observation of the breastbone section of each of experimental groups in which normal mice are combined administered with sOGP and rhG-CSF after administration of sOGP for 5 days alone. FIG. 3A is normal control group. FIG. 3B is the group administered by sOGP alone. FIG. 3C is the group co-administrated with sOGP and rhG-SCF. FIG. 3D is the group administrated with rhG-CSF alone.

FIG. 4 shows the pathologic observation of the spleen section of each of experimental groups in which normal mice are combined administered with sOGP and rhG-CSF after administration of sOGP for 5 days alone. FIG. 4A is normal control group. FIG. 4B is the group administered by sOGP alone. FIG. 4C is the group co-administrated with sOGP and r G-SCF. FIG. 4D is the group administrated with rhG-CSF alone.

FIG. 5 shows the effect of simultaneously administration of sOGP and rhG-CSF to normal mice on white blood cell (WBC) number before/after administration in different time.

#: the comparison of co-administration group and the group using rhG-CSF alone, p<0.0005.

### DETAILED DESCRIPTION OF INVENTION

After comprehensive and intensive researches, the inventors have found that, the co-administration of OGP and G-CSF has a synergistic effect, significantly enhancing the efficiency of G-CSF, thereby more efficiently improving the proliferation of hematopoietic cells (primarily granulocyte) and the recruitment of peripheral blood stem cell. On the basis of said discovery, the inventors completed this invention.

As used herein, the term “promoting hemopoiesis” or “hemopoiesis promotion” means that the co-administration of OGP and G-CSF improves the recruitment of peripheral blood stem cell, ameliorates diseases, or makes the patient recover from the decrease of peripheral blood neutrophilic granulocyte caused by radiotherapy lesion or chemotherapy drugs.

As used herein, the term “amino acid” means any of the 20 naturally occurring amino acids ( in three-letter code): Gly, Ala, Asp, Glu, Asn, Gln, Ser, Thr, Leu, Ile, Lys, Arg, Phe, Tyr, Trp, Pro, Cys, Met, His, Val, unless otherwise specified. The term includes D- and L-amino acids. Further, the term also includes synthetic amino acid, methylated amino acid, and allo-amino acid.

Granulocyte Colony-Stimulating Factor (G-CSF)

The G-CSF useful in the invention is from any resources, including natural and recombinant G-CSF, its analogs and active fragments, and the pharmaceutically acceptable salts thereof. The recombinant hG-CSF is particularly preferred. The commercial products of G-CSF include, but are not limited to, filgrastim, lenograstim, nartograstim, or the combination thereof.

The safe and efficient amount of G-CSF is usually 0.1-1000 ug/kg body weight, preferably 0.2-250 ug/kg body weight. For example, when administrated to human for various indications such as inducing the formation of peripheral blood before or after radiotherapy or chemotherapy and bone marrow transplantation, the dose of rhG-CSF is 0.4-100 ug per kg body weight. When administrated to mammal animals such as rats, the dose is 100-500 ug per kg body weight.

Osteogenic Growth Peptide or OGP

As used herein, the term “OGP” includes t he natural OGP polypeptide, synthetic OGP polypeptide, all OGP analogs, OGP-related peptides, and the pharmaceutically acceptable salts thereof.

The OGP useful in the invention includes all forms of the above OGP, especially the synthetic or recombinant OGP.

The preferred OGP is the human OGP having the amino acid sequence of ALKRQGRTLYGFGG.

Moreover, the OGP-related peptide is also useful in the invention. The preferred OGP-related peptide is derived from the C terminal of OGP and has the amino acid sequence of formula (I):
X1-X2-Y-X3-F-X4-X5-X6-X7 (I)
wherein X1 is amino, acetyl, acetylated amino acid or deaminated amino acid; each of X2 and X6 is independently none or a single amino acid, or is several amino acids or a peptide; each of X3, X4, and X5 is independently an amino acid; X7 is amino, carboxyl or hydroxyl, wherein the amino acid for X1-X6 is selected from the group consisting of Gly, Ala, Asp, Glu, Asn, Gln, Ser, Thr, Leu, Ile, Lys, Arg, Phe, Tyr, Trp, Pro, Cys, Met, His, Val;
Y is Tyr, F is Phe, and the length of OGP-related peptide is 5-15 amino acids.

It has been showed in the previous study that the OGP-related peptide of formula (I) has the OGP activity as long as the two active sites, Y and F, are retained. (See U.S. Pat. No. 5,814,610 and Chen Y C et al., J. Med. Chem. Apr. 11, 2002; 45(8):1624-32).

As to the method for producing OGP and OGP-related peptides, there is no special limitation. One can use solid or liquid chemical synthesis techniques, or gene recombination methodology, or enzyme processing.

A preferred method is to use conventional chemical synthesis techniques, such as solid or liquid process, to produce the OGP and OGP-related peptides useful in the invention. The solid synthesis is more preferred (See, e.g., Birr, C., Aspect of the Merrifield Peptide Synthesis, Springer-Verlag, Heidelberg, 1978; Stewart et al., Solid Phase Peptide Synthesis, 2nd. ed., Pierce Chem.co., Rockford, Ill., 1984; Barany, G. And Merrifield R. B. in The Peptides, Vol.2; Gross, E. & Meienhoffer J., eds., Academic Press, New York, pp3-284, 1979). Briefly, according to the designed certain amino acid sequence, the proper activators and condensation agents are firstly used to link the C-terminal of peptide chain protected by suitable protecting groups onto the solid supports. According to the different kinds of linked amino acid, one can select and use various solid supports for polypeptide synthesis, which include but are not limited to PEG, divinylbenzene crosslinked polystyrene, and polyacrylamide resin.

One can use any of known methods to convert the OGP produced by using the above techniques or recombinant DNA techniques into the pharmaceutically acceptable salts thereof. For example, one can use suitable acids, bases to make these polypeptides into proper salts according to the methods well known to the skilled in the art.

In the invention, the safe and efficient amount of OGP is usually 0.1 μg-100 mg per kg body weight, preferably 0.5 ug-10 mg per kg body weight.

Pharmaceutical Composition

The pharmaceutical composition of the invention comprises the following components:
(1) OGP and/or OGP-related peptide, or the pharmaceutically acceptable salts thereof,
(2) the acceptable amount of G-CSF or the pharmaceutically acceptable salts thereof,
(3) a pharmaceutically acceptable carrier,
wherein the molar ratio of OGP to G-CSF is from 0.25:1 to 100:1. Preferably, the molar ratio is from 1:1 to 20:1.

A preferred method for determining the amounts of the components is as follows: firstly, the amount of G-CSF is selected as a conventionally acceptable amount of G-CSF, and then the amount of OGP is determined based on the above molar ratio. Since OGP is a short peptide whose cost is low, the amount of OGP is usually higher than or equal to the amount of G-CSF so as to obtain the best ratio of efficiency to cost.

In addition to OGP and/or OGP-related peptide and rhG-CSF, the pharmaceutical composition of the invention may further comprise any of the drugs clinically used for haematogenesis such as GM-CSF, EPO, or the pharmaceutically acceptable salts thereof or the combination thereof. It may further comprise any of the drugs clinically used for improve the immunity, such as interleukin-2 ( IL-2), or the combination thereof, or their pharmaceutically acceptable salts.

In addition to the above components, the pharmaceutical composition of the invention may comprise any conventional solvents or preservatives. For the pharmaceutical composition in liquid form, there is no special limitation to pH range, which is usually from 4 to 8.5. Moreover, the pharmaceutical composition maybe made into lyophilized formulation.

The pharmaceutical composition of the invention may contain suitable carriers or diluents such as water, saline, isotonic glucose solution to prepare the formulations for parenteral administration such as solutions, injections, emulsions, nose drops, eye drops. The excipients or carriers such as starch, lactose, tale powder, sucrose, glucose or glycerol, liquid paraffin, liposome, albumin or gelatin can be added to make the formulations containing OGP and G-CSF for gastrointestinal administration such as suppositories, tablets, powders, granules, capsules or liposome encapsulated formulation. In addition to the active ingredients and suitable carriers and excipients, some auxiliary components can be added into these formulations, such as one or more diluents, fillers, emulsifying agents, preservatives, surfactants, absorption promoting agents, buffering agents, flavors and pigments.

As to the way of administration for the pharmaceutical composition of the invention, there is no special limitation. It can be administrated in any way compatible to the form of the preparation, e.g., subcutaneously or intramuscularly, or by infusion.

The pharmaceutical composition of the invention has the following applications:
(1) promoting the recruitment of peripheral blood stem cell in a donor for peripheral blood stem cell transplantation;
(2) treating the disorders caused by the decrease of peripheral blood neutrophilic granulocyte resulted from radiotherapy lesion or chemotherapy drugs;
(3) promoting the recovery of the peripheral blood leucocyte in bone marrow transplantation and facilitating the survival of donor cells;
(4) preventing the acute radiation sickness.

The inventors prepared the composition containing OGP and G-CSF and observed the effects on promoting the haematogenesis by co-administrating OGP a nd rhG-CSF i n the following experiments:
a) the effect on the number of peripheral blood leucocyte in normal mice by co-administrating OGP and rhG-CSF;
b) the effect on the number of peripheral blood lymphocyte in normal mice by co-administrating OGP and rhG-CSF;
c) the effect on the number of peripheral blood erythrocyte and thrombocyte in normal mice by co-administrating OGP and rhG-CSF;
d) the effect on the number and classification of bone marrow karyotes in normal mice by co-administrating OGP and rhG-CSF;
e) the pathologic observation on breastbone section of normal mice;
f) the effect on the weight of spleen in normal mice by co-administrating OGP and rhG-CSF;
g) the pathologic observation on spleen section of normal mice.

The results indicated that the co-administration of OGP and G-CSF significantly promoted the increase of peripheral blood leucocyte in normal mice and the number was increased by 2-3 folds compared to that in the mice administrated only the same dose of G-CSF and by 5-6 folds compared to that in the normal control. The co-administration of OGP and G-CSF significantly promoted the increase of peripheral blood lymphocyte in normal mice and the number was increased by 2-3 folds compared to that in the mice administrated only the same dose of G-CSF and by 3-4 folds compared to that in the normal control. Meanwhile, it promoted the enlargement of spleen in mice. The detection on karyote classification in spleen sections showed that the haematogenesis of spleen granulocyte was promoted. No abnormal proliferation of bone marrow was observed in the administration group during the experiments such as counting the bone marrow, detecting the bone marrow smear and detecting breastbone.

As to the mechanism of synergistic effects, the inventors deemed that, OGP might decrease the adhesive moleculars such as integrin on the surface of the hemopoietic stem cells or progenitor cells, thereby promoting the release of the stem cells and progenitor cells from bone marrow into the peripheral blood and promoting the extramedullary hemopoiesis of spleen so as to enhance the function of G-CSF. OGP may also influence the microenvironment of the bone marrow matrix, and act on cells in the bone marrow matrix so as to increase the expression of other hemopoietic cytokines or up-regulate the receptors for G-CSF, thereby enhancing the function of G-CSF. However, it would be appreciated that the present invention is limited by the above mechanism.

The studies of the invention indicated that the pharmaceutical composition containing sOGP and rhG-CSF could effectively promote the hemopoiesis without the side effects such as the abnormal proliferation of bone marrow. Further, this pharmaceutical composition may be used to treat diseases of immune system. Therefore, it has wide clinical application potential.

The invention is further illustrated by the following examples. These examples a re only intended to illustrate the invention, but not to limit the scope of the invention. For the experimental methods in the following examples, they are performed under routine conditions or as instructed by the manufacturers, unless otherwise specified.

### EXAMPLE 1

Synthesis of OGP and the Related Peptides with the Fmoc System or the Boc System

The OGP and the related peptides were synthesized by using the Fmoc system or the Boc system according to the methods disclosed in Chinese Application No. 99113596.2. For example, the synthesis of OGP with the Boc system was began with 0.32 mmol of BocQ-Pam-resin. The sequence was extended from the C terminus to the N terminus one by one according to the sequence of the polypeptide. The protective groups used for various Boc amino acids were Lys(CIZ), Arg(Tos), Thr(Bzl), Tyr(Brz), respectively. The condensation agent was DCCI, and the carboxyl group of each amino acid of which was activated by adding HOBT. Boc was removed by 50%TFA after the beginning of each cycle, then 10% DIEA was added for neutralization. The peptide chain was treated under 0° C. with dried hydrogen fluoride containing 5% p-cresol after it was synthesized. Then it was cleaved from the resin and the protective groups were removed simultaneously, extracted with 1N HCl, desalted with Sephadex G10 and lyophilized, thereby obtaining the crude peptide. Then, the crude peptide was separated and purified by chromatography with TSK HW-40F gel column. The analysis of the composition of amino acid and the determination of the purity were performed.

An OGP with an amino acid sequence of ALKRQGRTLYGFGG and multiple OGP-related peptides were obtained.

### EXAMPLE 2

The synergistic Effect of OGP and G-CSF on Promoting Haematogenesis

2.1 Materials

Osteogenesis growth peptide: the synthesized OGP (sOGP) prepared in Example 1.

Granulocyte colony stimulating factor: commercially available recombinant human granulocyte colony stimulating factor (rhG-CSF).

2.2 Methods

The clean grade of the Balb/c mice, weighed from 16 to 18 g, were used and randomly grouped, 7-10 mice per group. The tests were carried out in two batches.

Test 1

The mice were divided into 4 groups. When studying the synergistic effect of sOGP and rhG-CSF, sOGP was first administered for 5 days, then sOGP and rhG-CSF were administered simultaneously.
A group: the normal control group;
B group: the group administered with sOGP alone with a dose of 0.5 nmol per mouse;
C group: the group administered with sOGP in combination with rhG-CSF, wherein the dose of sOGP was 0.5 nmol per mouse, and the dose of G-CSF was 100 μg per kg body weight, and the molar ratio between them was about 5.5 to 1;
D group: the group administered with G-CSF alone with a dose of 100 μg per kg body weight.

The sOGP was administered daily to Group B from Day 1 to Day 13. As to the C group, sOGP was first administered for 5 days, then sOGP and G-CSF were co-administered for 8 days. As to the D group, rhG-CSF was administered from the sixth day and lasted for 8 days. The mice in each group were sacrificed on the 14th day.

The detection indexes were as follows:
(1) The counts of the peripheral blood leucocytes (WBC), the peripheral blood lymphocytes (LY), red blood cells (RBCs) and the change of platelet were determined for each group on the first day (no drug was administered), the 5th, 8th, 10th, 12th and 14th day.
(2) The counts of marrow karyotes (BMNC) of the mice in each group and the change of the classification of the marrow were determined on the 14^{th} day.
(3) The sections of the sternum of the mice in each group were determined on the 14^{th} day.
(4) The weights of the spleens of the mice in each group and the sections of the sleep were determined on the 14^{th} day.

Test 2

The mice were divided into 4 groups. When studying the synergistic effect of sOGP and rhG-CSF, they were administered simultaneously.
A group: the normal control group;
B group: the group administered with sOGP alone with a dose of 0.5 nmol per mouse;
C group: the group administered with sOGP in combination with rhG-CSF, wherein the dose of sOGP was 0.5 nmol per mouse, and the dose of G-CSF was 100 μg per kg body weight, and the molar ratio between them was about 5.5 to 1;
D group: the group administered with G-CSF alone with a dose of 100 μg per kg body weight.

The sOGP was administered daily to the B group from Day 1 to Day 10. As to the C group, sOGP and G-CSF were co-administered for 10 days. As to the D group, rhG-CSF was administered from the first day and lasted for 10 days. The mice in each group were sacrificed on the eleventh day.

The detection indexes were as follows:
(1) The counts of the peripheral blood leucocytes of the mice in each group were determined on the first day (no drug was administered), the 3rd, 5th, 7th, 9th and 11th day.
(2) The counts of marrow karyotes (BMNC) of the mice in each group were determined on the 11^{th} day.
(3) The weights of the spleens of the mice in each group were determined on the 11^{th} day.

2.3 Statistical Treatment

All of data were represented by x±SE. The t-test was used for the experimental data.

2.4 Results

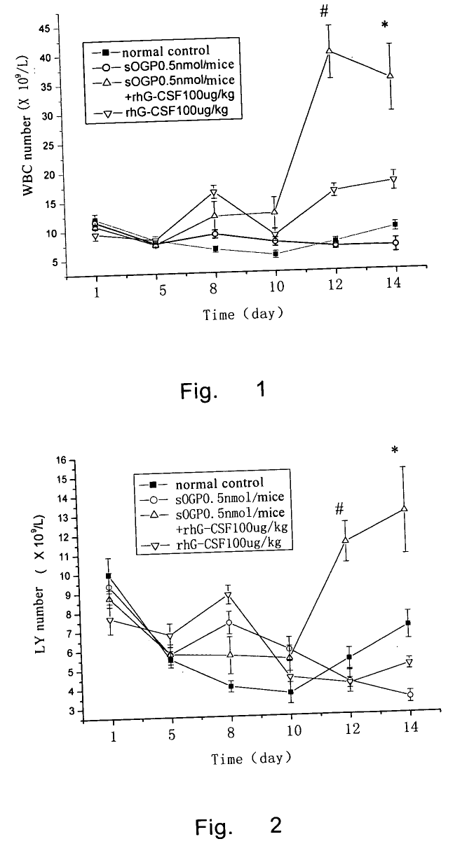

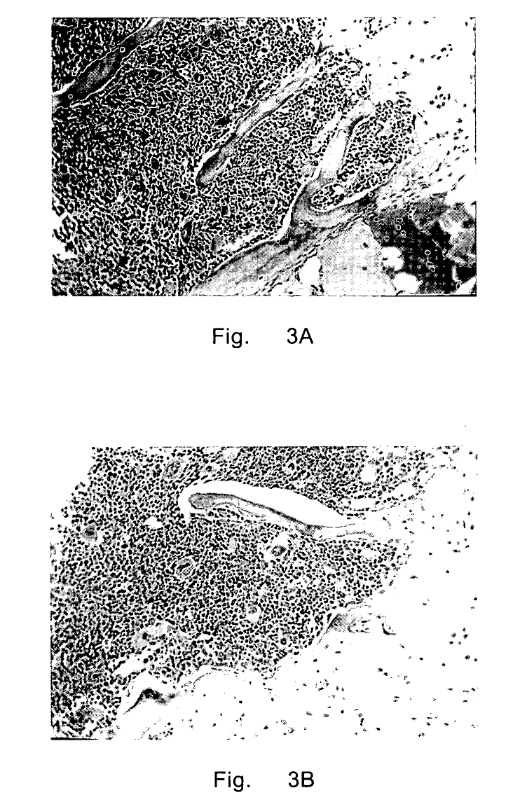

The results were indicated in FIGS. 1-5 and Tables 1-5.

Table 1 showed the influence on the ratio of the peripheral blood granulocyte and lymphocyte, and the number of red blood cells (PBCs) and platelet (PLC) produced in the normal mice administered with sOGP in combination with rhG-CSF after administration of sOGP for 5 days.

TABLE 1

| | | | | red blood | |
|---|---|---|---|---|---|
| | % leucocyte | | leucocyte | cell | platelet |
| | granulocyte | lymphocyte | (×10⁶/ml) | (×10⁹/ml) | (×10⁶/ml) |
| control | 28.26 ± 1.67 | 71.74 ± 1.67 | 9.77 ± 0.81 | 7.40 ± 0.24 | 502.29 ± 14.66 |
| sOGP | 48.90 ± 1.83 | 51.10 ± 1.83 | 6.56 ± 0.42 | 7.02 ± 0.10 | 500.88 ± 11.20 |
| SOGP + rhG- | 63.06 ± 1.20 | 36.94 ± 1.20 | 35.1 ± 5.67 | 6.74 ± 0.19 | 495.71 ± 56.64 |
| CSF | | | | | |
| G-CSF | 70.39 ± 1.54 | 29.61 ± 1.54 | 17.59 ± 1.59 | 6.68 ± 0.10 | 505.43 ± 35.34 |

Table 2 showed the influence on total number of the marrow karyote and the change of the classification thereof of the thighbone in the normal mice co-administrated with sOGP and rhG-CSF after administration of sOGP for 5 days.

TABLE 2

| | marrow (cell number/thighbone) (%) | | | |
|---|---|---|---|---|
| | | | sOGP + rhG- | |
| | Control | sOGP | CSF | rhG-CSF |
| Red blood cell line | | | | |
| primitive erythroblasts | 1.57 ± 0.37 | 1.13 ± 0.30 | 1.38 ± 0.38 | 1.63 ± 0.32 |
| early erythroblast | 4.57 ± 0.48 | 3.37 ± 0.38 | 3.25 ± 0.49 | 2.50 ± 0.33 |
| intermediate erythroblast | 12.14 ± 0.86 | 13.75 ± 1.56 | 11.38 ± 0.71 | 12.13 ± 1.92 |
| late erythroblast | 22.14 ± 1.52 | 19.63 ± 2.23 | 19.63 ± 1.82 | 16.00 ± 2.11 |
| granulocyte cell line | | | | |
| primitive granulocyte | 0.86 ± 0.27 | 0.75 ± 0.25 | 0.63 ± 0.18 | 0.63 ± 0.36 |
| promyelocyte | 2.29 ± 0.36 | 1.50 ± 0.27 | 1.75 ± 0.37 | 1.88 ± 0.44 |
| myelocyte, metamyelocyte | 11.43 ± 0.90 | 13.75 ± 1.37 | 14.25 ± 1.54 | 11.00 ± 1.00 |
| matured granulocyte | 21.43 ± 1.76 | 24.25 ± 1.61 | 24.75 ± 2.38 | 30.83 ± 2.77 |
| acidophil or basophil granulocyte | 1.71 ± 0.29 | 1.63 ± 0.92 | 1.50 ± 0.32 | 1.63 ± 0.42 |
| megakaryocyte | 1.29 ± 0.18 | 1.00 ± 0.27 | 0.75 ± 0.25 | 1.13 ± 0.23 |
| lymphocyte cell line | | | | |
| lymphocyte, plasma cell | 20.57 ± 1.51 | 19.25 ± 1.65 | 20.75 ± 1.42 | 19.38 ± 2.00 |
| karyote/thighbone(×10⁷) | 1.98 ± 0.07 | 1.82 ± 0.11 | 1.99 ± 0.11 | 1.90 ± 0.13 |

Table 3 showed the influence on the weight of the spleen of the normal mice produced by the co-administration of sOGP and rhG-CSF after administration of sOGP for 5 days.

TABLE 3

| | Control | sOGP | sOGP + rhG-CSF | rhG-CSF |
|---|---|---|---|---|
| spleen weight (g) | 0.136 ± 0.004 | 0.114 ± 0.005 | 0.262 ± 0.011 | 0.252 ± 0.007 |

Table 4 showed the influence on the total number of the marrow karyote of the thighbone in the normal mice co-administered with sOGP and rhG-CSF.

TABLE 4

| | Control | sOGP | sOGP + rhG-CSF | rhG-CSF |
|---|---|---|---|---|
| karyote number/ | 1.74 ± 0.08 | 1.74 ± 0.06 | 1.88 ± 0.08 | 1.75 ± 0.12 |
| thighbone(×10⁷) | | | | |

Table 5 showed the influence on the weight of the spleen of the normal mice by administering sOGP and rhG-CSF simultaneously. #: The co-administration group was compared with the group administered with rhG-CSF alone, p<0.05.

TABLE 5

| | Control | sOGP | sOGP + rhG-CSF | rhG-CSF |
|---|---|---|---|---|
| spleen weight (g) | 0.112 ± 0.004 | 0.102 ± 0.005 | 0.300 ± 0.021 | 0.244 ± 0.017 |

2.5 Discussion

1. The influences produced by co-administrating sOGP and rhG-CSF after administration of sOGP for 5 days included on the following aspects: the counts of the peripheral blood leucocytes; the classification of the leukocytes, i.e., the counts of the granulocytes and lymphocytes and the ratio between them; the counts of the red blood cells and platelet; the marrow karyotes and the classification of the marrow cells; the observation of the sections of sternum; the observation of the sections of mice spleen; and the weight of mice spleen.

(1) It could be seen from FIG. 1 that, after the 11^{th} day, the count of peripheral blood leucocytes (WBC) in the group co-administered with sOGP and rhG-CSF was significantly higher than those in the other groups. It was about 2.5 times of that in rhG-CSF group (p<0.0005), and 5 times of that in the normal control group (p<0.00005). After the 13^{th} day, the count of peripheral blood leucocytes in the group co-administered with sOGP and rhG-CSF was significantly higher than those in the other groups and specifically, about 2 times of that in the rhG-CSF group (p<0.05), and 4 times of that in the normal control group (p<0.001).

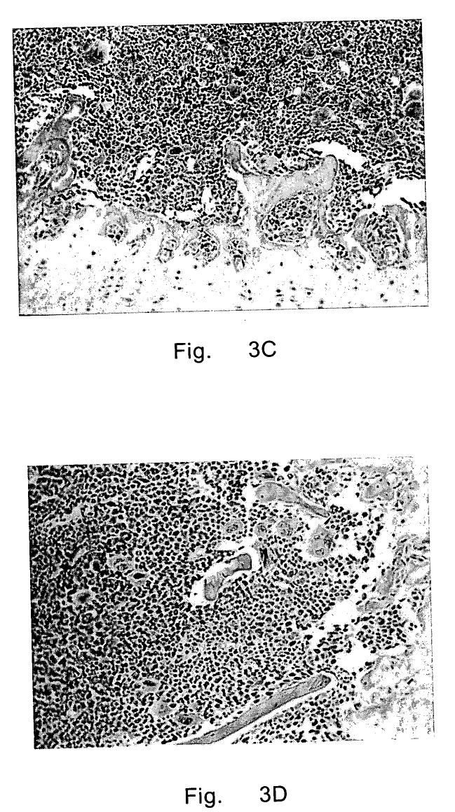

It could be seen from FIG. 2 that, after the 11^{th} and the 13^{th} days, the count of the peripheral blood lymphocytes was significantly higher than those of the other groups, specifically, about 2 times of that in the rhG-CSF group (p<0.05) and about 2 times of that in the normal group (P<0.05-0.0005).

As to the influence on the classification of the leucocyte, it could be seen from Table 1 that in the group co-administered with sOGP and rhG-CSF, the ratio of the granulocytes to the leucocytes was higher than those of the control group and the sOGP group, but lower than that of rhG-CSF group, and the ratio of the lymphocytes to the leukocytes was lower than those in the control group and the sOGP group, but higher than that in the rhG-CSF group.

As to the influence on the counts of the red blood cells and the platelet, no significant difference could be found among the groups.

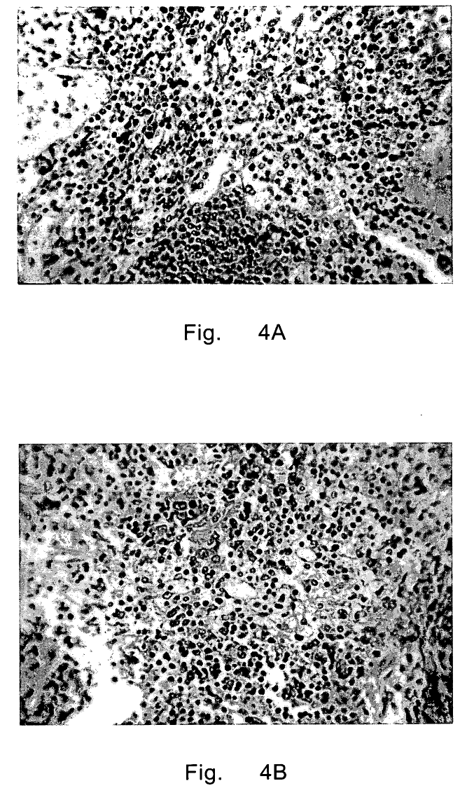

(2) It could be seen from FIG. 3 that the bone marrow cells of the negative control group (FIG. A) were normally proliferated. The ratios among the three cell lines were normal and no specific pathological change was found.

The bone marrow cells of the group administered with sOGP alone (FIG. B) were normally proliferated, the ratios among the three cell lines were normal and no specific pathological change was found.

The bone marrow cells of the group co-administered with sOGP and rhG-CSF (FIG. C) were normally proliferated, the ratios among the three cell lines were normal and no specific pathological change was found.

As to the group administered with rhG-CSF alone (FIG. D), the proliferation of the bone marrow cells and the ratios among the three cell lines were normal. However, the ratio of the matured granulocyte cell line to the granulocyte cell line was a little high. This may be a reactive proliferation, not a pathological change.

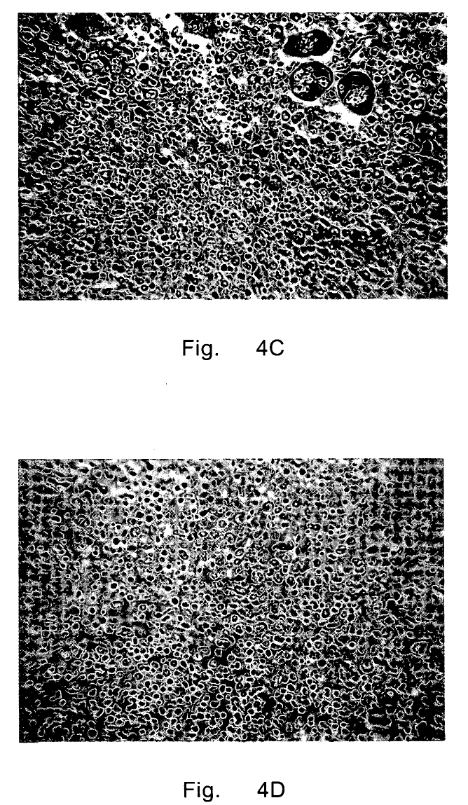

(3) It could be seen from FIG. 4 that the white matter of the negative control group (FIG. A) was normal. There were few hemopoietic cells in the red matter, and the ratio between the granulocytes and the red blood cells was about 0.5 to 1. Most of the cells in the granulocyte cell line were the immature cells. The matured granulocytes comprised about 25% of the granulocytes. The megakaryocyte cell line was slightly proliferated.

The white matter of the group administered with sOGP alone (FIG. B) was normal. There are few hemopoietic cells in the red matter, and the ratio between the granulocytes and the red blood cells was about 1 to 1. The matured granulocytes comprised about 30% of the granulocytes.

The white matter of the group co-administered with sOGP and rhG-CSF (FIG. C) was mildly or moderately shrunk, the red matter was proliferated moderately and contained a lot of hemopoietic cells. The ratio between the granulocytes and the red blood cells was about 1.75:1. The megakaryocyte cell line was moderately proliferated. The matured granulocytes in the granulocyte cell line comprised about 60% of the granulocytes.

The white matter of the group administered with rhG-CSF alone (FIG. D) was lightly to moderately shrunk, the red matter was proliferated moderately and contains a lot of hemopoietic cells. The ratio between the granulocytes and the red blood cells was about 2:1. The megakaryocyte cell line was moderately proliferated. The matured granulocytes in the granulocyte cell line comprised about 60% of the granulocytes.

It could be seen from this test that the haematogenesis inside the spleen and outside the marrow (the granulocyte, red blood cell and megakaryocyte cell lines) was significant in Groups C and D. Group B was slightly insignificant. According to the pathological morphology thereof and the ratios of the cells in each stage of each system, the haematogenesis function of the bone marrow resulted in responsive proliferation other than cancerous proliferation.

(4) It could be seen from Table 2 that the marrow karyotes in the thighbone of mice in the group co-administered with sOGP and rhG-CSF was not significantly different from the other three groups, and no abnormal proliferation of the marrow was observed. As compared to the control group, the count of the promyelocyte in the co-administration group was less, while the counts of the myelocyte, metamyelocyte and the matured granulocyte was increased, indicating that the maturation of the promyelocyte could be expedited and it was possible to facilitate the release of the relatively matured granulocyte from the bone marrow into the peripheral blood, thus enhancing the count of the leucocyte in the peripheral blood.

(5) It could be seen from Table 3 that the spleen weights of the mice in the group co-administered with sOGP and rhG-CSF and the group administered with rhG-CSF alone were significantly higher than in the other two groups. And, the weight of the group co-administered with sOGP and rhG-CSF tended to be higher than that in the group administered with rhG-CSF alone, but it was not significantly different, which indicated that it was possible to increase the count of the leucocyte in the peripheral blood by facilitating the haematogenesis of the spleen.

2. The influence of co-administration of sOGP and rhG-CSF on the counts of the leucocyte, the lymphocytes in the peripheral blood of mice, and the weight of the spleen of the mice.

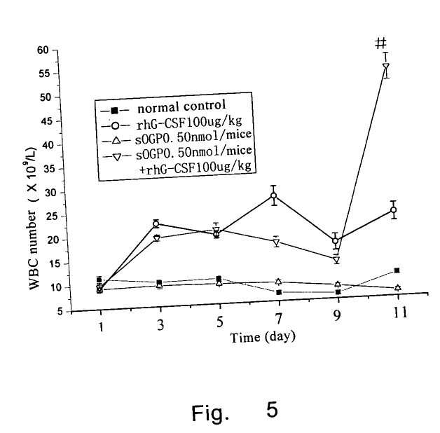

(1) It could be seen from FIG. 5 that after 10 days of co-administration of sOGP and rhG-CSF, the count of the leucocyte in the peripheral blood was significantly higher than those in the other groups. It was about 2.3 times of that in the rhG-CSF group (P<0.0005) and 5.3 times of that in the normal control group (P<0.00005).

(2) It could be seen from FIG. 4 that there was no significant difference between the count of the marrow karyotes in thighbone of the mice in the group co-administered with sOGP and rhG-CSF and those obtained in the other three groups. And no abnormal proliferation of the bone marrow was observed.

(3) It could be seen from Table 5 that the spleen weights of the mice in the group co-administered with sOGP and rhG-CSF and the group administered with rhG-CSF alone were significantly higher than those in the other two groups. The weight of the group co-administered with sOGP and rhG-CSF was significantly higher than that obtained in the group administered with rhG-CSF alone (P<0.05).

### EXAMPLE 3

The Synergistic Effect of OGP and G-CSF on Promoting Haematogenesis

This example studied the synergistic effects when using different molar ratios between the osteogenesis growth peptide and G-CSF.

The example was carried out basically according to Test 2 in Example 2, except that the amounts of OGP were changed, i.e., the molar ratios between the osteogenesis growth peptide and G-CSF were 0.5:1, 1:1, 10:1 or 20:1.

As a result, the synergistic effects produced by the osteogenesis growth peptide and G-CSF on the promotion of haematogenesis were also observed.

All the documents cited herein are incorporated into the invention as reference, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A pharmaceutical composition comprising a safe and efficient amount of osteogenic growth peptide or OGP, a safe and efficient amount of granulocyte colony-stimulating factor or G-CSF and a pharmaceutically acceptable carrier, wherein the molar ratio of OGP to G-CSF is from 0.25:1 to 100:1.

2. The pharmaceutical composition of claim 1 further comprising a component selected from the group consisting of GM-CSF, EPO, IL-2, or the combination thereof.

3. The pharmaceutical composition of claim 1 wherein the OGP is selected from the group consisting of human OGP, OGP-related peptide, their pharmaceutically acceptable salts, and the combination thereof.

4. The pharmaceutical composition of claim 1, wherein the molar ratio of OGP to G-CSF is from 1:1 to 20:1.

5. The pharmaceutical composition of claim 1, wherein the safe and efficient amount of OGP is 0.1 ug-100 mg per kg body weight, and the safe and efficient amount of G-CSF is 0.1-1000 ug/kg body weight.

6. The pharmaceutical composition of claim 1, wherein the formulation of the pharmaceutical composition is injection, or lyophilized powder.

7. The pharmaceutical composition of claim 3, wherein the human OGP has the amino acid sequence of ALKRQGRTLYGFGG; and
the OGP-related peptide is derived from the C terminal of OGP and has the amino acid sequence of formula (I):
X1-X2-Y-X3-F-X4-X5-X6-X7 (I)
wherein X1 is amino, acetyl, acetylated amino acid or deaminated amino acid; each of X2 and X6 is independently none or a single amino acid, or is several amino acids or a peptide; each of X3, X4, and X5 is independently an amino acid; X7 is amino, carboxyl or hydroxyl, wherein the amino acid for X1-X6 is selected from the group consisting of Gly, Ala, Asp, Glu, Asn, Gln, Ser, Thr, Leu, Ile, Lys, Arg, Phe, Tyr, Trp, Pro, Cys, Met, His, Val;
Y is Tyr, F is Phe, and the length of OGP-related peptide is 5-15 amino acids.

8. A method for preparing a pharmaceutical composition, comprising the following steps:
mixing the OGP, G-CSF and a pharmaceutically acceptable carrier, thereby obtaining the pharmaceutical composition, wherein the molar ratio of OGP to G-CSF is from 0.25:1 to 100:1.

9. The method of claim 8 wherein the molar ratio of OGP to G-CSF is from 1:1 to 20:1.

10. The method of claim 8 wherein the formulation of the pharmaceutical composition is injection, or lyophilized powder.
